(19) United States
(12) Patent Application Publication
(10) Pub. No.: US 2012/0225823 A1
(43) Pub. Date: Sep. 6, 2012

(54) Allosteric Modulation of the Dopamine Transporter Protein for the Treatment of HIV-1 Induced Neurologic Dysfunction

(75) Inventors: Rosemarie M. Booze, Columbia, SC (US); Charles F. Mactutus, Columbia, SC (US); Jun Zhu, Irmo, SC (US)

(73) Assignee: UNIVERSITY OF SOUTH CAROLINA, Columbia, SC (US)

(21) Appl. No.: 13/454,209

(22) Filed: Apr. 24, 2012

Related U.S. Application Data
(63) Continuation of application No. 12/620,170, filed on Nov. 17, 2009, now abandoned.
(60) Provisional application No. 61/115,306, filed on Nov. 17, 2008.

(51) Int. Cl. A61K 38/16; A61P 25/36; A61P 25/00

(52) U.S. Cl. 514/18.1

(57) ABSTRACT

A method for increasing dopaminergic neurotransmission in a mammal in need of such treatment is provided. The method comprises disrupting Tat-DAT binding in the mammal.

### CROSS REFERENCE TO RELATED APPLICATION

The present application is a continuation of U.S. application Ser. No. 12/620,170 having a filing date of Nov. 17, 2009 which is based on U.S. Provisional Application Ser. No. 61/115,306 having a filing date of Nov. 17, 2008. Applicants claim priority to and benefit of all such application and incorporate all such applications herein by reference.

### GOVERNMENT SUPPORT CLAUSE

The present invention was developed with funding from the National Institutes of Health under awards DA011337, DA09160, DA013137, and HD43680. Therefore, the government retains certain rights in this invention.

### BACKGROUND

Recent estimates indicate that there are 30-40 million people infected with human immunodeficiency virus (HIV) over the world. Injection drug use is a high risk behavior associated with the transmission of HIV, and accounts for approximately one-third of the total number of acquired immunodeficiency syndrome (AIDS) cases in the United States. HIV-1 trans-activator of transcription (Tat) protein is essential for efficient viral replication and can be released from acutely infected cells as a biologically active protein. Tat protein is thought to play a crucial role in pathogenesis of HIV-1-associated dementia. This Tat-associated neurotoxicity may, in part, reflect an apparent dysfunction of the dopaminergic system.

The dopamine (DA) transporter (DAT) is a specific marker for DA terminals and plays a critical role in a number of neurological disorders, including drug abuse. The re-uptake of DA through the DAT is the primary mechanism that regulates extracellular DA concentrations, and terminates DA actions at the synapse. DATs are high-affinity targets for cocaine and amphetamine, both of which are highly addictive and widely abused substances. Clinical studies using positron emission tomography have found that HIV-1 infected patients with HAD had a significant reduction in DAT density in the putamen and ventral striatum.

HIV-1 Tat is a nonstructural viral protein of 101 amino acids, encoded from two separate exons. Peptides derived from the first exon of Tat, including Tat₄₆₋₆₀, Tat₃₇₋₇₂ and Tat₃₁₋₆₁, have been found to cause neurotoxicity. Intrastriatal injections of Tat damage both efferent and afferent projections of the striatum, including nigrostriatal DA neurons. An in vivo microdialysis study recently reported that intra-striatal infusion of Tat decreased K⁺-evoked DA levels in rat, suggesting that the in vivo exposure to Tat causes alterations in DA transmission. In addition, intrastriatal Tat decreased amphetamine-evoked DA overflow and a depletion of DA levels seven days after local injections. The protein and mRNA levels of tyrosine hydroxylase (TH) were decreased in rat dopaminergic PC12 cells in the presence of Tat. These results indicate Tat interferes with the biosynthetic pathway, the production, and the release of DA. Therefore, Tat-induced neurotoxicity may reflect damage of dopaminergic system with functional Parkinsonian features and pathology. In fact, several publications have reported clinical outcomes of Parkinsonian symptoms and depletion of substantia nigra DA neurons in HIV-infected patients.

In addition, it has been shown that Tat protein decreases [³H]DA uptake into rat striatal synaptosomes. Intra-accumbal Tat₁₋₇₂ increases acute cocaine-induced locomotor activity and attenuates repeated cocaine-induced behavioral sensitization. Rats injected with Tat and systemic methamphetamine showed a reduction in [¹²⁵I]RTI-121 binding to DAT in striatum and significantly decreased levels of TH 24 hours later. In rat fetal midbrain primary culture cells, incubation with Tat protein caused reductions in the [³H]WIN 35,428 binding and increases in Tat-induced neurotoxicity when co-incubated with GBR12909, a selective DAT inhibitor. Taken together, these results suggest that Tat protein-induced neurotoxicity, at least in part, is mediated through reduced DAT activity, and that a combination of Tat protein and abused drugs enhances neurotoxicity in a synergistic manner.

To date the mechanistic interactions between the Tat and DAT proteins have not been subject to a detailed pharmacological assessment. In the present disclosure, the inhibitory effects of Tat on DAT function and the radioligand binding sites have been determined, including dose response and time course studies. [³H]WIN 35,428 binding has been shown to share pharmacological identity with the DA uptake carrier and to be part of the cocaine binding domain. In accordance with the present disclosure, it has been determined that recombinant Tat₁₋₈₆ protein differentially inhibited specific [³H]DA uptake and the binding of [³H]WIN 35,428 and [³H]GBR 12935 in rat striatal synaptosomes, which has potential implication for therapeutic approaches to HIV-associated cognitive-motor disorders.

Molecular determinants of Tat neurotoxicity are located within the 1-72 sequence encoded by the first tat exon. The second exon-encoded part of the full-length Tat sequence is non-essential for the direct neurotoxicity. Amino acid residues 22-38 comprise highly conservative cysteine-rich domain of Tat. Experimental evidence indicates the important role of the cysteine-rich domain in mechanisms of Tat toxicity. The discovery of lower cytotoxic potential of HIV-1 virus clade C, which is thought to be attributed to the mutation of cysteine 31 in the 1-101 version of Tat protein expressed in this HIV-1 subtype, has fueled the interest to the role of individual cysteines located in the cysteine-rich domain for the ability of Tat to cause neurodegeneration.

### SUMMARY

Objects and advantages of the invention will be set forth in part in the following description, or may be obvious from the description, or may be learned through the practice of the invention.

In accordance with certain embodiments of the present disclosure, a method for increasing dopaminergic neurotransmission in a mammal in need of such treatment is provided. The method comprises disrupting Tat-DAT binding in the mammal.

In still other embodiments of the present disclosure, a method for increasing dopaminergic neurotransmission in a mammal in need of such treatment is provided. The method comprises disrupting Tat-DAT binding in the mammal by altering protein Tat cys22.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1. Pharmacological profiles of [³H]DA uptake in the presence of Tat₁₋₈₆, Tat_{Δ31-61}, Tat cys22, or GBR 12909. Striatal synaptosomes were preincubated with various concentrations of Tat₁₋₈₆, Tat_{Δ31-61}, Tat cys22, or GBR 12909 (0.1 nM-10 μM) at 34° C. for 15 min followed by the addition of [³H]DA (0.1 μM final concentration) for 10 min. Tat_{Δ31-61} and Tat cys22 were used as negative controls. GBR 12909 was used as a positive control. Data are expressed as mean±S.E.M. as percentage of control (CON) values (25205±2065 dpm) from 5 independent experiments performed in duplicate. Nonspecific [³H]DA uptake was determined in the presence of 10 μM nomifensine. All curves were best fitted to a single class of binding site and generated by nonlinear regression.

FIG. 2. Time course of Tat effects on [³H]DA uptake into rat striatal synaptosomes. Striatal synaptosomes pooled from two rats were used for all samples. For a single experiment, aliquots of synaptosomes were preincubated with Tat₁₋₈₆ (1 or 10 μM) or the same volume of assay buffer (control) at 34° C. for the indicated times (1 to 60 minutes), followed by the addition of [³H]DA (0.1 μM final concentration) at 34° C. for 10 min. Four pairs of independent inhibition experiments were performed at each time, and the raw data were analyzed as matched pairs. Data are expressed as mean±S.E.M. as percentage of the respective control values. *p<0.05 compared to the controls.

FIG. 3. Effect of Tat on kinetic analysis of [³H]DA uptake into rat striatal synaptosomes. Kinetic analysis of the synaptosomal [³H]DA uptake was determined in the absence (control) or presence of Tat₁₋₈₆ at concentration of 1 μM (A) or 10 μM (B). Striatal synaptosomes were preincubated with or without Tat at 34° C. for 15 min followed by the addition of one of eight mixed concentrations of the [³H]DA as described herein In parallel, nonspecific uptake at each concentration of the mixed [³H]DA (in the presence of 10 μM nomifensine, final concentration) was subtracted from total uptake to calculate DAT-mediated uptake. The Vₘₐₓ and Kₘ values were estimated by fitting the data to the Michaelis-Menten equation and represent the means from five independent experiments±S.E.M. Inset, Eadie-Hofstee transformation of the same kinetic data. *^{#}p<0.05, compared to the respective control values.

FIG. 4. Tat protein inhibited [³H]WIN35,428 and [³H]GBR 12935 binding in a time-dependent manner. A. Aliquots of synaptosomes were incubated with Tat₁₋₈₆ (1 or 10 μM) and 5 nM [³H]WIN 35,428 on ice for the indicated times (1 to 60 minutes). B. Tat₁₋₈₆ (1 or 10 μM) was incubated with striatal synaptosomes and 5 nM [³H]GBR 12935 at room temperature for the indicated times. Four pairs of independent inhibition experiments were performed at each time, and the raw data were analyzed as matched pairs. Nonspecific binding for [³H]WIN 35,428 and [³H]GBR 12935 was determined in the presence of 30 μM cocaine and 30 μM GBR 12909, respectively. Data are expressed as mean percentage±S.E.M. as of the respective control (in the absence of Tat) values (n=3). *p<0.05 compared to the controls.

FIG. 5. Pharmacological profiles of Tat₁₋₈₆, WIN35,428 or GBR 12909 inhibition of [³H]WIN 35,428 and [³H]GBR 12935 binding in rat striatal synaptosomes. For a single experiment, striatal synaptosomes were incubated with the indicated concentrations of Tat₁₋₈₆, WIN35,428 or GBR 12909 in the presence of 5 nM [³H]WIN 35,428 or 5 nM [³H]GBR 12935. A. Specific [³H]WIN 35,428 competition curves expressed as mean±S.E.M. percent control (CON) values (7926±499 dpm) from 5 independent experiments performed in duplicate. B. Specific [³H]GBR 12935 competition curves are expressed as mean±S.E.M. percent of control (CON) values (8500±340 dpm) from 5 independent experiments performed in duplicate. Nonspecific binding for [³H]WIN 35,428 and [³H]GBR 12935 was determined in the presence of 30 μM cocaine and 30 μM GBR 12909, respectively. All curves were best fitted to a single class of binding site.

FIG. 6. Characterization of [³H]WIN 35,428 binding in striatal synaptosomes in the absence and presence of Tat. Striatal synaptosomes from two rats were pooled, and half of the pooled sample was used for [³H]WIN 35,429 binding in the presence of Tat₁₋₈₆, and the other half of the pooled sample was used for control. Saturation isotherm for [³H]WIN 35,428 binding to striatal synaptosomes was determined in the absence (control) or presence of Tat₁₋₈₆ at the concentration of 0.2 μM (A) or 1 μM (C). Bₘₐₓ (pmol/mg protein) and K_{d} (nM) values for control and Tat are presented. Scatchard transformations of same data are presented for control and Tat₁₋₈₆ at the concentration of 0.2 μM (B) or 1 μM (D). Nonspecific binding for [³H]WIN 35,428 was determined in the presence of 30 μM cocaine. Data were best fit to a single class of binding site and are presented as means±S.E.M. from 4 independent experiments. *, ^{#}p<0.05, compared to the respective control values.

FIG. 7. Characterization of [³H]GBR 12935 binding in striatal synaptosomes in the absence and presence of Tat. Saturation isotherms for [³]MGBR12935 binding to striatal synaptosomes were determined in the absence (control) or presence of Tat₁₋₈₆. For a single experiment, striatal synaptosomes from two rats were pooled, and half of the pooled sample was used for [³H]GBR 12935 binding in the presence of Tat, and other half of the pooled sample was used for control. Bₘₐₓ and K_{d} values from three independent experiments are presented for control and Tat₁₋₈₆ at the concentration of 0.2 μM (A), 1 μM (B) or 10 μM (C). Nonspecific binding for [³]MGBR12935 was determined in the presence of 30 μM GBR12909. Data were best fit to a single class of binding site and are presented as means±S.E.M. from 4 independent experiments. *p<0.05, compared to the control values.

FIG. 8. Effects of Tat on [³H]leucine and [³H]DA uptake into striatal synaptosomes. A. Striatal synaptosomes were preincubated with various concentrations of Tat₁₋₈₆, Tat_{Δ31-61}, or leucine at the concentration as indicated at 34° C. for 15 min followed by the addition of [³H] leucine (1 μM final concentration) for 10 min. Data are expressed as mean±S.E.M. as percentage of control (CON) values (24808±1289 dpm) from 5 independent experiments performed in duplicate. B. Synaptosomes were preincubated with various concentrations of Tat₁₋₈₆ (0.1 nM-10 μM) at 34° C. for 5 min followed by the addition of [³H]DA (0.1 μM final concentration) or [³H]leucine (1 μM final concentration) for 4 min. Nonspecific uptake for [³H]leucine and [³H]DA was determined in the presence of 10 mM L-lysine and 10 μM nomifensine, respectively. Data are expressed as mean±S.E.M. as percentage of control (CON) values (26000±1300 dpm) from 4 independent experiments performed in duplicate. All curves were best fitted to a single class of binding site and generated by nonlinear regression. *p<0.05, compared to the control values.

FIG. 9. Neuronal cell viability changes in hippocampal cell cultures induced by the original and cysteine 22-substituted Tat 1-86 clade B. (A) The dose-response of decreased neuronal cell viability in primary rat fetal hippocampal cell cultures exposed to Tat 1-86 or Cys 22 Tat 1-86. The graph shows the decrease in Live/Dead ratios produced by different doses of recombinant Tat 1-86 after 48 hours of treatment. Data presented as mean values, n of sister cultures analyzed=8-15 per each Tat 1-86 concentration. *-marks significant (P<0.05) differences in Live/Dead ratios between cultures treated with Cys22 Tat 1-86 and vehicle-treated controls. **-marks significant (P<0.05) differences in Live/Dead ratios between cultures treated with Tat 1-86 and cultures exposed to the same dose of Cys22 Tat 1-86. (B) The time course of the changes in neuronal cell viability in primary rat fetal hippocampal cell cultures exposed to Tat 1-86 or Cys 22 Tat 1-86. The graph represents relative (compared to non-treated controls) changes in Live/Dead ratios following the addition of 50 nM Tat 1-86 or 50 nM Cys 22 Tat 1-86. Individual measurements were carried out in 4-8 sister cultures (wells of the 96-well plate) per each time point and the experiment was repeated three times to ensure the reproducibility of the results. Data presented as mean values. *-marks incubation time points when significant (P<0.05) differences in Live/Dead ratios between cultures treated with Cys22 Tat 1-86 and vehicle-treated controls have been observed. **-marks incubation time points when significant (P<0.05) differences in Live/Dead ratios between cultures treated with Tat 1-86 and Cys22 Tat 1-86 have been observed.

FIG. 10. Direct interactions of Tat 1-86 and Cys22 Tat 1-86 with hippocampal cells and following changes in cell viability and caspase activation. Binding/uptake of Tat 1-86 or Cys22 Tat 1-86 by cultured rat fetal hippocampal neurons. (A) Images show the specific Tat (or Cys22 Tat) immunoreactivity in rat fetal hippocampal cell cultures exposed to 50 nM Tat 1-86 or 50 nM Cys 22 Tat 1-86 for 2 hours. (B) Western blots show the specific Tat immunoreactivity in cell lysates 2 hours after the addition of either 50 nM Tat or 50 nM of Cys22 Tat 1-86 to the cell culture medium. (C) The graph shows amounts of Tat 1-86 or Cys22 Tat 1-86 per well specifically absorbed by hippocampal cells during the first 2 hours of treatment. Data presented as mean values±SEM (n=3 per each time point). Neurotoxic effects of the transient exposure of primary rat fetal hippocampal cell cultures to Tat 1-86 or Cys 22 Tat 1-86. (D) Cell cultures were exposed to 50 nM dose of Tat 1-86 or Cys22 Tat 1-86 for different time periods ranging from 1 min to 2 hours. After the end of exposure, the cell culture medium was replaced with a fresh portion of medium without Tat or Cys22 Tat. The graph shows Live/Dead ratios determined in hippocampal cell cultures 48 hours after different time periods of transient exposure to original and Cys22-substituted Tat 1-86. Data presented as mean values, n of sister cultures analyzed=7-12 per each time point. *-marks time periods of transient exposure to Tat 1-86, which were sufficient to induce a significant (P<0.05) decrease in Live/Dead ratios compared to vehicle-treated controls. The caspase 9 and caspase 3/7 activities in primary rat fetal hippocampal cell cultures after the exposure to Tat 1-86 or Cys 22 Tat 1-86. (E) Representative images show results of the detection of the SR-LEND (red) fluorescent signal attributed to activated caspase 9 in neurons treated for 2 hours and results of the detection of the FAM-DEVD (green) fluorescent signal attributed to activated caspase 3/7 in neurons treated for 24 hours (B) with either 50 nM Tat 1-86 or 50 nM Cys 22 Tat 1-86 . Cultures were co-stained with Hoechst (blue fluorescence).

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure describes mechanistic interactions between the HIV-1 Tat protein and DAT protein in rat striatum using [³H]DA uptake, [³H]WIN 35,428 and [³H]GBR 12935 binding assays. The present disclosure demonstrates time- and concentration-dependent effects of Tat in inhibiting [³H]DA uptake into striatal synaptosomes. Importantly, the data presented here provide novel evidence that the Tat-induced decrease in DAT activity is accompanied by distinctly different changes in both the binding sites of [³H]WIN 35,428 and [³H]GBR 12935, and in the regulatory properties of DAT. These changes have profound implications for understanding dysfunctional DA regulation via DAT alteration, which may underlie the neurochemical basis of HIV-induced neuronal dysfunction and synergistic neurotoxicity of HIV with drugs of abuse. Further, targeting of cysteines that form Tat metal-binding site may be considered as one of the therapeutic strategies to simultaneously block transactivating and neurotoxic Tat abilities.

The competition of Tat with [³H]DA uptake revealed that micromolar Tat concentrations were required for the inhibitory effects, which corresponded to the potency for the Tat inhibition of [³H]GBR 12935 binding. In contrast, the Kᵢ value for [³H]WIN 35,428 binding is 3-fold more potent than that for [³H]DA uptake, suggesting that Tat interacts with different sites on DAT protein. The deletion mutant proteins, Tat_{Δ31-61} and Tat cys22 showed no inhibitions in [³H]DA uptake, suggesting that the these amino acids play a crucial role in interaction with the DAT. The Tat protein used in the present disclosure contains 86 amino acids encoded by two exons. Peptides derived from the first exon of Tat, including Tat₄₆₋₆₀, Tat₃₇₋₇₂, and Tat₃₁₋₆₁, have been shown to cause neurotoxicity in vitro. Thus, the implication from the results described herein is that Tat protein-induced neurotoxicity may be at least partially mediated via direct and specific inhibition of DAT protein.

Time course studies in accordance with the present disclosure reveal that the Tat inhibited [³H]DA uptake and the binding of either [³H]WIN 35,428 or [³H]GBR 12935 in a time-dependent manner. Compared to the Tat effects on the binding of the two radioligands, the effects of Tat on [³H]DA uptake were more delayed. For example, 1 μM Tat inhibited [³H]DA uptake by 5% at 5 min, whereas the [³H]WIN 35,428 and [³H]GBR 12935 binding were inhibited by 40% and 25%, respectively at the 5 min time. Also, at the 5 min time point, Tat (1 μM)-induced inhibition in the binding of [³H]WIN 35,428 and [³H]GBR 12935 reached a plateau, whereas Tat effects on [³H]DA uptake exhibited a maximal inhibition at 15 min and remained until 60 min. It has been previously demonstrated that cocaine-induced increases in [³H]DA uptake were more rapid than that observed for the [³H]WIN 35,428 binding, using human DAT-stably transfected N2A cells. It is possible that markedly distinct mechanisms are at play in the processes presented here versus those caused by exposure to cocaine, which results from trafficking DAT from intracellular DAT to cell surface.

In accordance with the present disclosure, a separate experiment was performed to determine if the Tat-induced decrease in the DAT Vₘₐₓ is reversible. After 15 min incubation with Tat, the Tat-induced decrease in Vₘₐₓ of [³H]DA uptake returned to the control level, suggesting reversibility of DAT function following a short exposure to Tat. Thus, the effects of Tat protein on DA system appear to be comprised of two distinct phases: (1) a rapid and reversible phase where decreases in [³H]DA uptake are attributable to a loss of binding sites of [³H]WIN 35,428 and [³H]GBR 12935; (2) a later persistent phase, presumably associated with a loss of dopaminergic terminals, in which Tat-induced decreases in DAT function and protein are not reversible.

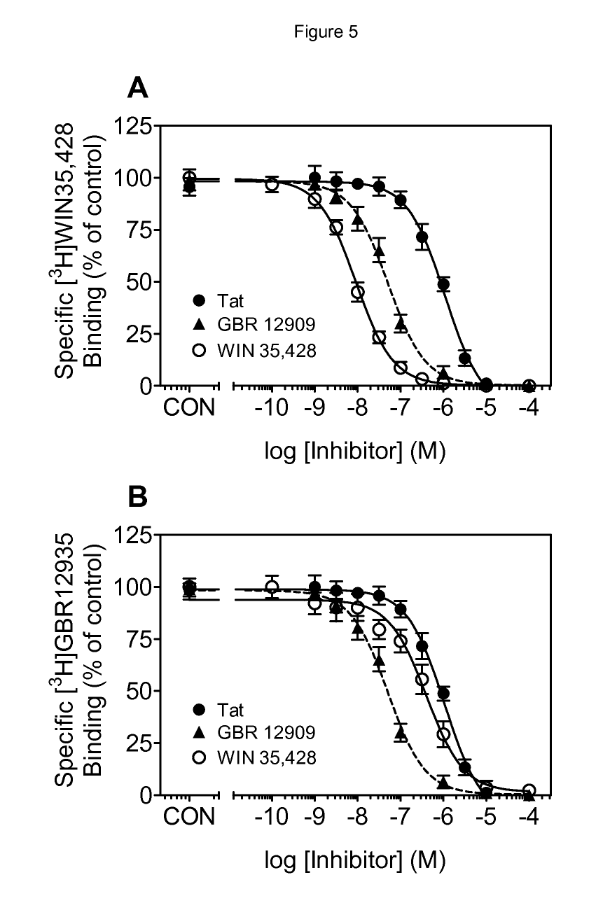

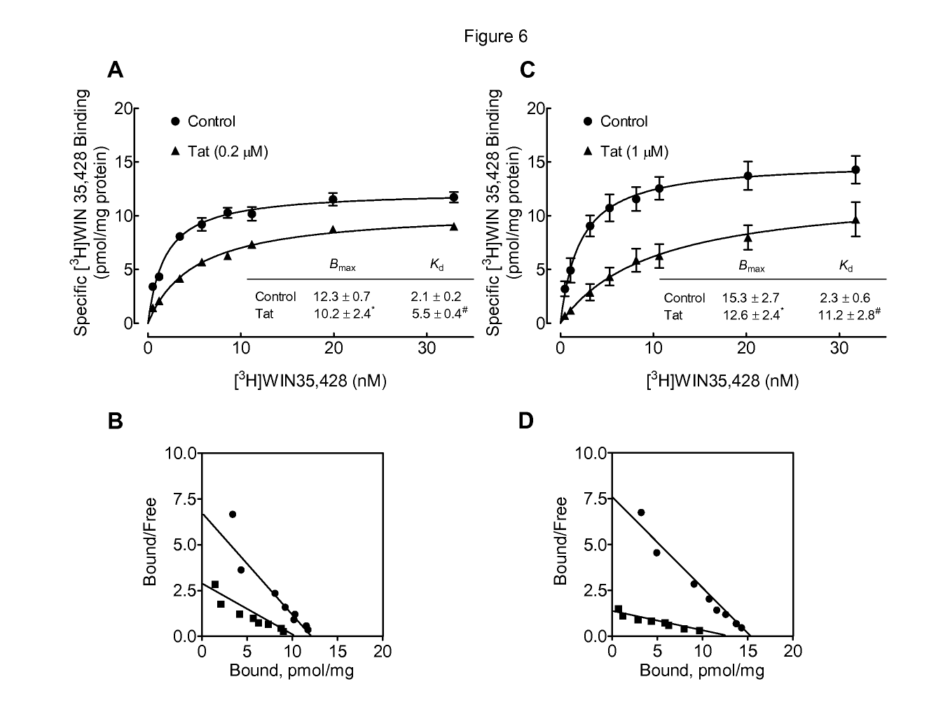

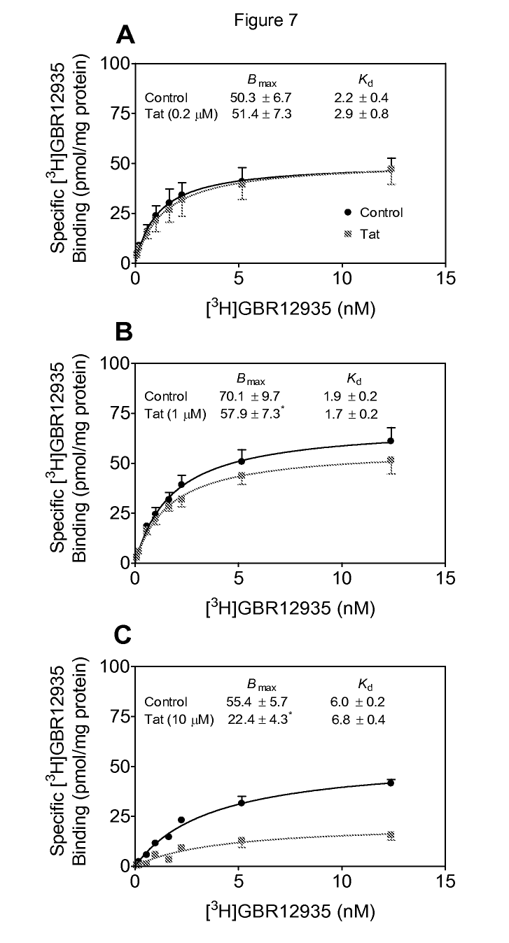

Several lines of evidence from the present disclosure indicate that Tat protein interacts at selective binding sites on the DAT, which was indicated by the differential results from the [³H]WIN 35,428 and the [³H]GBR12935 binding assays. First, the Kᵢ value for Tat inhibition in the [³H]GBR12935 binding is 4-fold higher than that in the [³H]WIN 35,428 binding (Table 1). Second, Tat dose-dependently inhibited the Bₘₐₓ values of the binding of either [³H]WIN 35,428 or [³H]GBR12935, however, Tat increased the K_{d} value in the [³H]WIN 35,428 binding but not in the [³H]GBR12935 binding (FIGS. 6 and 7). Third, WIN 35,428 displaced [³H]WIN35,428 binding with high affinity and Hill coefficient, compared to that in the displacement experiments of the [³H]GBR 12935 binding (FIG. 5). WIN35,428 and GBR12935 are useful for labeling elements of the DAT; GBR 12935 labels the classic DA uptake site in rodent brain and binds a piperazine acceptor site, whereas WIN35,428 binds the cocaine binding sites. For example, the Bₘₐₓ for [³H]GBR 12935 in the control group is about 5 times higher than observed with the [³H]WIN35,428 binding (FIGS. 6 and 7). The [³H]WIN 35,428- and the [³H]GBR 12935-labeled DAT binding sites likely have some overlap. However, DAT sites labeled by these ligands may reflect different aspects of the functional DA uptake process, because [³H]GBR 12935 and [³H]WIN 35,428 do not appear to bind the same functional form/state of DAT in COS-7 expressed human DAT, whereas in rodent brain the two radioligands appear to have different binding characteristics as well. In addition, the different conditions used for measuring the binding of the two radioligands (differentially optimized for maximal binding) could also play a role in the divergent results.

A number of independent measures in the present disclosure demonstrated Tat-induced decreases in DAT function and radioligand binding sites. The decrease in the DAT function seemed to be due to a reduction of total DAT protein, based on the decrease in the Bₘₐₓ of [³H]WIN 35,428 and [³H]GBR 12935. In addition, other experiments of the present disclosure indicate that the effect of Tat at the low concentration was specific to the DAT because Tat (1 μM) caused no changes in [³H]leucine uptake. Exposure to a high Tat concentration (10 μM) decreased [³H]leucine uptake, suggesting that Tat produces global changes in endocytosis rather than an effect specific to DAT. However, the 10 μM Tat concentration is most likely not physiological; actual Tat levels in the brain from HIV infected human are reported to be less than 140 picomolar. It has previously been reported that alteration of DAT Vₘₐₓ without changing the DAT B. was related to a redistribution of DAT protein in the cytosolic versus plasma membrane expression, which was confirmed by cell surface biotinylation assay. The magnitude of the Tat-induced decrease was greater in the [³H]DA uptake than that in either the binding of [³H]WIN 35,428 or [³H]GBR 12935. For example, Tat (1 μM) caused a 26% decrease recorded in DAT Vₘₐₓ, and a ˜18% decrease in the B. of [³H]WIN 35,428 and [³H]GBR 12935. These differences probably reflect that the [³H]DA uptake assay only measures a large amount of cell surface DAT that has been reported to be about 60% of the total DAT in rat striatum. Thus, assessing the potential redistribution of DAT protein in cytoplasmic pools and cell surface expression caused by exposure to Tat would likely be beneficial.

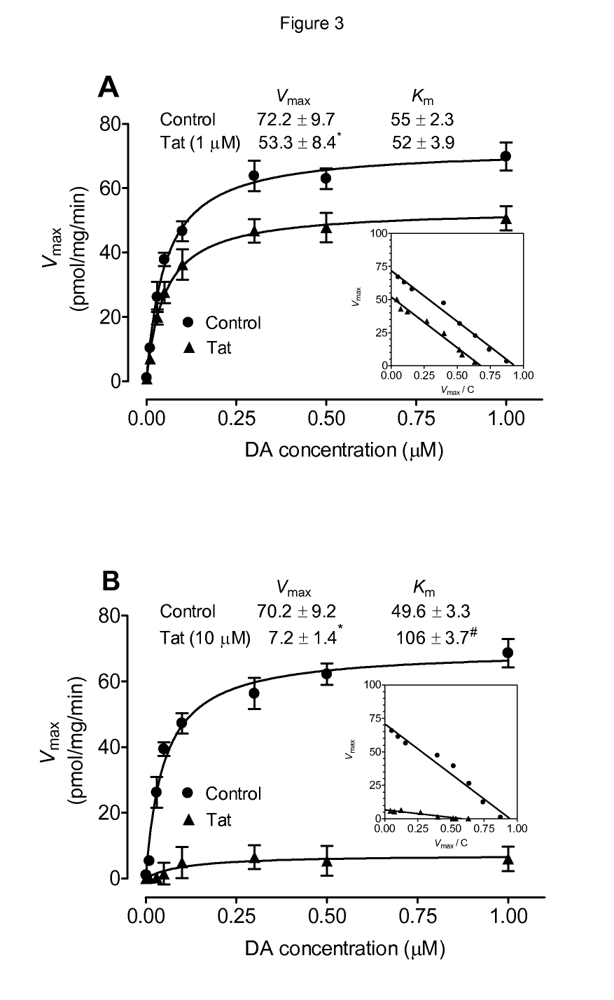

As an alternative explanation to DAT trafficking away from the neuronal cell surface, the results of the present disclosure can be interpreted in terms of Tat-induced allosteric modulation of DAT function. Tat at 0.2 μM decreased the Vₘₐₓ (26%) without changing the Kₘ value (FIG. 3A), but 10 μM Tat decreased the Vₘₐₓ by 90% and increased the apparent Kₘ value (FIG. 3B), consonant with Tat allosterically modulating DAT function in a dose-dependent manner. In addition, Tat dose-dependently decreased the Bₘₐₓ of [³H]WIN 35,428 and increased the apparent K_{d} value (FIG. 6), deviating from a purely competitive mechanism of inhibition by Tat. Thus, Tat may act as an allosteric modulator of DAT rather than as either a reuptake inhibitor or a substrate-type releaser, such as cocaine and amphetamine. The different kinetics for inhibition of [³H]GBR 12935 binding by Tat are consonant with the different binding characteristics reported for the two radioligands in rodent brain. A recent study reported that the novel allosteric modulators of the DAT, SoRI-20040, SoRI-20041 and SoRI-2827, which decreased the Vₘₐₓ and Bₘₐₓ values of [³H]DA uptake and [¹²⁵I]RTI-55 binding and increased the apparent Kₘ and K_{d} values in a dose-dependent manner. In general, drugs interact with transporter proteins in two ways, either as a reuptake inhibitor or as a substrate. There is growing interest in an additional layer of complexity of how compounds can interact with transporters: allosteric modulation. In accordance with the present disclosure, such allosterism could represent a novel therapeutic potential or, underlie a neurobiological mechanism of dysfunction of DAT reported in the patients with HIV infection, consonant with a potential susceptibility to drug intake in those patients. The present disclosure can facilitate the development of therapeutic programs for HIV infection in humans. For instance, the present disclosure describes the effect of the mutation of the cysteine 22, which is essential for Zn⁺²-chelating and trans-activation abilities of HIV-1 Tat, with Gly on neurotoxic properties of the recombinant Tat 1-86 (clade B) in the primary culture of rat fetal hippocampal neurons.

The present disclosure can be better understood with reference to the following example.

### EXAMPLE

### Materials and Methods

Materials. D-Glucose was purchased from Aldrich Chemical Co, Inc. (Milwaukee, Wis.). [³H]DA (3,4-ethyl-2[N-³H]dihydroxyphenylethylamine; specific Activity, 31 Ci/mmol), [³H]WIN 35,428 (−)-3β-(4-fluorophentl)-tropan-2β-carboxylic acid methyl ester tartrate, 85 Ci/mmol), [³H]GBR 12935 (GBR 12935 [propylene-2,3-³H], specific activity 43.5 Ci/mmol), and L-[³H]Leucine (specific activity 140 Ci/mmol) were purchased from Perkin Elmer Life Sciences (Boston, Mass.). Recombinant HIV-1 trans-activator of transcription (Tat₁₋₈₆) protein and its mutant protein, Tat cys22 (cystine 22 was substituted to glycine) were purchased from DIATHEVA (Fano, ITALY). A deletion of mutant of Tat protein devoid of amino acids 31-61 (Tat_{Δ31-61}) protein was provided by the department of Neurology, Johns Hopkins Hospital (Baltimore, Md.). L-Ascorbic acid, GBR 12909, WIN 35,428, L-leucine, L-lysine, bovine serum albumin, pyrocatechol, α-D-glucose, HEPES, nomifensine maleate, pargyline hydrochloride, and sucrose were purchased from SigmaAldrich (St. Louis, Mo.). All other chemicals were purchased from Fisher Scientific (Pittsburgh, Pa.).
Animals. Adult male Sprague-Dawley rats (200-225 g, body weight) were obtained from Harlan Inc. (Indianapolis, Ind.) and housed in standard polyurethane cages with free access to food and water. The colony room was maintained at 22±2° C. and 45±10% humidity, with a 12 h:12 h light-dark cycle (lights on 0700 EST). The experimental procedures conformed to the 1996 NIH Guide for the Care and Use of Laboratory Animals.
Synaptosomal Preparation. Striata from individual rats were homogenized in 20 ml of ice-cold 0.32 M sucrose containing 5 mM NaHCO₃ (pH 7.4) with 16 up-and-down strokes using a Teflon pestle homogenizer (clearance approximately 0.003 inch). The resulting crude synaptosomal preparation was centrifuged at 2000 g for 10 min at 4° C., and the resulting supernatants were centrifuged at 20,000 g for 15 min at 4° C. The resulting pellets were resuspended in 5.0 ml of ice-cold Krebs-Ringer-HEPES assay buffer for [³H]DA uptake assay (125 mM NaCl, 5 mM KCl, 1.5 mM MgSO₄, 1.25 mM CaCl₂, 1.5 mM KH₂PO₄, 10 mM D-glucose, 25 mM HEPES, 0.1 mM EDTA, 0.1 mM pargyline, and 0.1 mM L-ascorbic acid, saturated with 95% O₂/5% CO₂, pH 7.4). Protein concentration was determined by the Bradford protein assay using bovine serum albumin as the standard (BIO-RAD).
[³H]DA Uptake Assay. [³H]DA uptake was determined using a method described in Zhu J, Green T, Bardo M T, and Dwoskin L P (2004) Environmental enrichment enhances sensitization to GBR 12935-induced activity and decreases dopamine transporter function in the medial prefrontal cortex, Behav Brain Res 148:107-117, which is incorporated by reference herein. Assays were performed in duplicate with a final volume of 250 μl. Aliquots of striatal synaptosomes (30 μl containing 20 μg protein) were added to the tubes containing 170 μl assay buffer and 25 μl of one of nine concentrations of Tat, Tat_{Δ31-61,} Tat cys22 or GBR12909 (final concentration 0.1 nM-10 μM,). GBR 12909 was used as a positive control for these experiments. Tat_{δ531-61} was chosen as a negative control based on a previous study showing that the region of the 31-61 amino acids plays an important role in the cellular uptake of Tat. Tat cys22 was another negative control for these experiments; a single mutation in the cystine 22 plays a critical role in the structural integrity of the activating domain of Tat protein. For the Tat dose response study, samples were incubated at 34° C. for 15 min in an oxygenated metabolic shaker. For the time course study, assay tubes were incubated at 34° C. for a range of time (1, 5, 15, 30, 45 and 60 min). Subsequently, 25 μl of 0.1 μM [³H]DA (final concentration) was added to each tube and the incubation was continued at 34° C. for 10 min. The reuptake of [³H]DA into rat striatal synaptosomes is in a linearity manner during 2-10 min period (data not shown). The reactions were terminated by the addition of 3 ml of ice-cold assay buffer. Samples were filtered through Whatman GF/B glass fiber filters, presoaked with assay buffer containing 1 mM pyrocatechol. Filters were washed 3 times with 3 ml ice-cold assay buffer containing 1 mM pyrocatechol using a Brandel cell harvester (Model M-48; Biochemical Research and Development Laboratories Inc., Gaithersburg, Md.). Pyrocatechol (Catechol) is a Catechol-O-methyltransferase (COMT) inhibitor. The COMT along with monoamine oxidases (MAO-A and -B) are the major mammalian enzymes involved in the metabolic degradation of DA. In the current example, pyrocatechol (1 mM) was included in the DA uptake assay buffer to prevent the degradation of [³H]DA during the processes of washes and harvesting. Radioactivity was determined by liquid scintillation spectrometry (Model TRI-CARB 2900TR, PerkinElmer Instruments, Shelton, Conn.).

To determine whether Tat-induced inhibition of [³H]DA uptake was the result of an alteration in either the maximal velocity (Vₘₐₓ) or Michaelis-Menten constant (Kₘ) of [³H]DA uptake, kinetic analyses were conducted in the absence or presence of Tat. To generate saturation isotherms, duplicate assay tubes were prepared as described above for the inhibition assays except that the concentration of DA added to each tube was varied. A fixed concentration of [³H]DA (500,000 dpm/tube) was isotopically diluted with one of eight concentrations of unlabeled DA (1.0 nM -1 μM, final DA concentrations). Synaptosomal samples were incubated with each concentration of [³H]DA in the absence or presence of the concentration of Tat (1 μM) that produced half-maximal inhibition (IC₅₀) of [³H]DA uptake in the inhibition assays. Nonspecific uptake was determined in duplicate at each [³H]DA concentration by including 10 μM nomifensine in the assay buffer. Kinetic parameters (Vₘₐₓ and Kₘ) were determined using GraphPad Prism 4.0 (GraphPad Software, Inc., San Diego, Calif.).

[³H]WIN 35,428 Binding Assay. To determine whether Tat-induced inhibition of [³H]DA uptake into striatal synaptosomes was the result of a direct interaction with DAT, the ability of Tat to inhibit [³H]WIN 35,428 binding in striatal synaptosomes was examined. [³H] WIN 35,428 binding was determined using a method described in Zhu J, Bardo M T, Bruntz R C, Stairs D J and Dwoskin L P (2007) Individual differences in response to novelty predict prefrontal cortex dopamine transporter function and cell surface expression, Eur J Neurosci 26: 717-728, which is incorporated by reference herein. Synaptosomes were prepared as described above, and the pellets were resuspended in 5.0 ml of ice-cold sodium-phosphate buffer (2.1 mM NaH₂PO₄, 7.3 mM Na₂HPO₄. 7H₂O, 320 mM sucrose, pH 7.4). The sucrose-phosphate buffer for [³H]WIN35,439 binding assay was utilized. There are two types of assay buffers for [³H]WIN35,439 binding, i.e., sucrose-phosphate buffer and Tris-NaCl buffer. The sucrose-phosphate buffer contains lower sodium ion concentration (˜30 mM) compared to that (100 mM) in Tris-NaCl buffer. It is believed that sucrose-phosphate buffer is more suitable to P2 membrane preparation and that Tris-NaCl buffer is commonly used in crude membrane preparation. Since WIN35,428 is an analog of cocaine, the binding assay in the sucrose-phosphate buffer is similar to that used originally for cocaine binding (Coffey and Reith, 1994). The presence of sucrose in the binding assay also increases the [³H]WIN35,439 binding to prevent a reduction in binding due to hypotonic media.

For the competitive inhibition experiment and time course study, assays were performed in duplicate in a final volume of 250 μl. Aliquots of the striatal synaptosomes (25 μl) were added to the assay tubes containing 25 μl of [³H]WIN 35,428 (5 nM, final concentration) and assay buffer (170 μl), 25 μl containing buffer or one of nine concentrations (1 nM-10 μM, final concentration) of Tat, WIN35,428 or GBR 12909. To determine whether Tat-induced inhibition of [³H]WIN 35,428 was the result of alterations in the maximal number of binding sites (Bₘₐₓ) or affinity (K_{d}) for these radioligands, kinetic analysis of [³H]WIN 35,428 binding was conducted.

To generate saturation isotherms, striatal synaptosomes from two rats were pooled, and half of the pooled sample was used for the [³H]WIN 35,429 binding in the presence of Tat₁₋₈₆ (1 μM) and the other half of the pooled sample was used for control (in the absence of Tat₁₋₈₆). This concentration of Tat was based on the results from the inhibition assays. Duplicate assay tubes were prepared as described above for the inhibition assays. Striatal synaptosomes were added to the assay tubes containing one of the eight concentrations of [³H] WIN 35,428 (0.5 to 30 nM, final concentration) in the absence (control) or presence of Tat₁₋₈₆ (0.2 or 1 μM). Assay tubes were incubated on ice for 2 h. In parallel, nonspecific binding at each concentration of [³H] WIN 35,428 (in the presence of 30 μM cocaine, final concentration) was subtracted from total binding to calculate the specific binding.

For the time course experiments, assay tubes were incubated on ice for a range of times (1, 5, 15, 30, 45 and 60 min). Nonspecific binding was determined in the presence of 30 μM cocaine. Assays were terminated by rapid filtration onto Whatman GF/B glass fiber filters, presoaked for 2 h with assay buffer containing 0.5% polyethylenimine, using the Brandel cell harvester. Filters were rinsed three times with 3 ml of ice-cold assay buffer. Radioactivity remaining on the filters was determined by liquid-scintillation spectrometry (Model TRI-CARB 2900TR, PerkinElmer Instruments, Shelton, Conn.).

[³H]GBR 12935 Binding Assay. The methods used to assay [³H]GBR 12935 binding are described in Zhu J, Green T, Bardo M T, and Dwoskin L P (2004) Environmental enrichment enhances sensitization to GBR 12935-induced activity and decreases dopamine transporter function in the medial prefrontal cortex, Behav Brain Res 148:107-117. Briefly, synaptosomes were prepared as described above, and resulting pellets following 20,000 g for 15 min were resuspended in 5.0 ml of ice-cold assay buffer (118 mM NaCl, 4.8 mM KCl, 2.5 mM CaCl₂, 1.2 mM MgCl₂, 25 mM HEPES, 10 mM D-glucose, pH 7.4). For the competitive inhibition experiment and time course study, assays were performed in duplicate in a final volume of 250 μl. Aliquots of striatal synaptosomes (25 μl) were added to assay tubes containing 25 μl of [³H]GBR 12935 (5 nM, final concentration), and assay buffer (170 μl), 25 μl containing buffer or 1 of 9 concentrations (1 nM-10 μM, final concentration) of Tat, WIN35,428 or GBR 12909. Saturation experiments of [³H]GBR 12935 binding were conducted in the absence or presence of Tat. To generate saturation isotherms, duplicate assay tubes were prepared as described above for the inhibition assays, except that one of the increased concentrations of [³H]GBR 12935 binding (0.5 to 30 nM, final concentration) was added to each tube in the absence or presence of Tat (1 μM). All procedures for the experiments were conducted at room temperature, except for the Tat solution, which was prepared with ice cold assay buffer. An equal amount of ice cold buffer was added into all assay tubes in the absence of Tat. Assay tubes were incubated at room temperature for 1 h. For the time course experiments, assay tubes were incubated at room temperature for a range of time (1, 5, 15, 30, 45 and 60 min). Nonspecific binding was determined in the presence of 30 μM GBR 12909. Assays were terminated by rapid filtration onto Whatman GF/B glass fiber filters, presoaked for 2 h with assay buffer containing 0.5% polyethylenimine, using Brandel cell harvester. Filters were rinsed three times with 3 ml of ice-cold assay buffer.
[³H]Leucine Uptake Assay. The potential inhibitory effect of Tat on electrochemical gradients was assessed using [³H]leucine uptake assay. The uptake assay was performed in duplicate with a final volume of 250 μl. Aliquots of striatal synaptosomes (30 μl) containing 20 μg protein were preincubated with a single concentration (25 μl) of Tat, Tat_{Δ31-61} or leucine and assay buffer (125 mM NaCl, 5 mM KCl, 1.5 mM MgSO₄, 1.25 mM CaCl₂, 1.5 mM KH₂PO₄, 10 mM glucose, 25 mM HEPES, 0.1 mM EDTA, 0.1 mM pargyline, and 0.1 mM L-ascorbic acid, saturated with 95% O₂/5% CO₂, pH 7.4). Tat_{Δ31-61} and leucine were chosen as negative and positive controls, respectively. Assay tubes were placed in an oxygenated metabolic shaker at 34° C. for 15 min. Subsequently, 1 μM [³H]leucine (final concentration, 25 μl) was added to each tube and incubation was continued for 10 min at 34° C. in the metabolic shaker. The competitive inhibition experiments for [³H]leucine and [³H]DA uptake were performed in the absence or presence of Tat (0.1 nM-10 μM final concentration) with a preincubation limited to 5 min, followed by adding 1 μM [³H]leucine or 0.1 μM [³H]DA (final concentration) for 4 min. Nonspecific uptake for [³H]leucine and [³H]DA was determined in the presence of 10 mM L-lysine and 10 μM nomifensine, respectively. Incubation was terminated by the addition of 3 ml of ice-cold assay buffer, followed by immediate filtration through Whatman GF/B glass fiber filters. Filters were washed 3 times with 3 ml ice-cold assay buffer using a Brandel cell harvester.
Data Analysis and Statistics. Data are presented as mean values±S.E.M., and n represents the number of independent experiments for each treatment group. For Tat-induced inhibition of [³H]DA uptake, [³H]WIN 35,428 and [³H]GBR 12935 binding, data are expressed as a percentage of control, i.e., specific [³H]DA uptake, [³H]WIN 35,428 or [³H]GBR 12935 binding in the absence of Tat. IC₅₀ values for Tat-induced inhibition in specific [³H]DA uptake or in specific binding of [³H]WIN 35,428 and [³H]GBR 12935 were determined from inhibition curves by nonlinear regression analysis using a one site model with variable slope. Inhibition curves were generated using 4 independent experiments with Tat. Kᵢ values were calculated according to the Cheng-Prusoff equation Kᵢ=IC₅₀/(1+L/K_{d}). Kₘ for [³H]DA uptake was defined as 30 nM from a pilot experiment (data not shown). The K_{d} value for [³H]WIN 35,428 and [³H]GBR 12935 binding was defined as 3 and 1.5 nM, respectively from a preliminary experiment (data not shown). Kinetic parameters (Bₘₐₓ, Vₘₐₓ, Kₘ or K_{d}) of [³H]DA uptake, [³H]WIN 35,428 and [³H]GBR 12935 binding were determined from saturation curves by nonlinear regression analysis using a one site model with variable slope. Experiments involving exposure to a single concentration (1 or 10 μM) of Tat with multiple time points were analyzed using separate one-factor ANOVAs, followed by simple comparison tests. In those experiments involving comparisons between two paired samples, paired Student's t tests were used to determine the ability of Tat to alter the kinetic parameters (Kₘ and Vₘₐₓ for [³H]DA uptake; K_{d} and Bₘₐₓ for [³H]WIN 35,428 and [³H]GBR 12935 binding compared to control (the absence of Tat); log transformed values of Kₘ or K_{d} were utilized for these statistical comparisons. All statistical analyses were performed using SPSS (standard version 16.0, Chicago, Ill.) and differences were considered significant at p<0.05.

### RESULTS

Tat Protein Inhibits [³H]DA Uptake into Rat Striatal Synaptosomes.

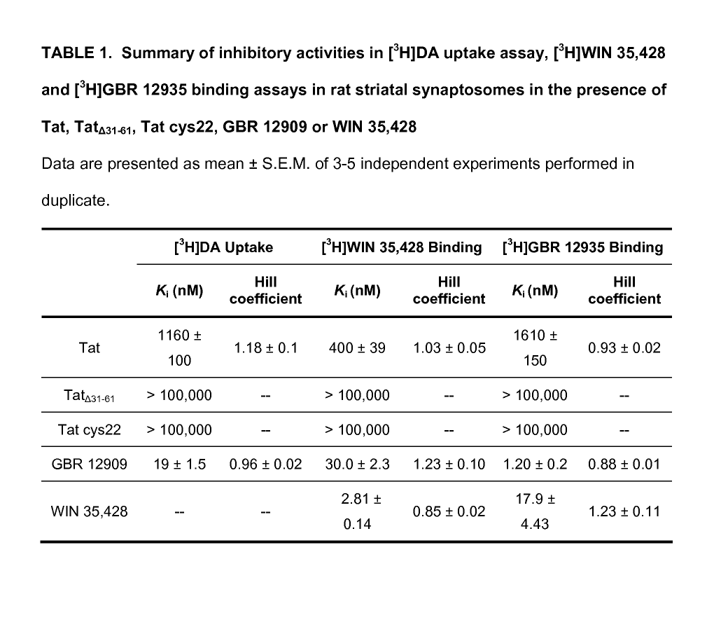

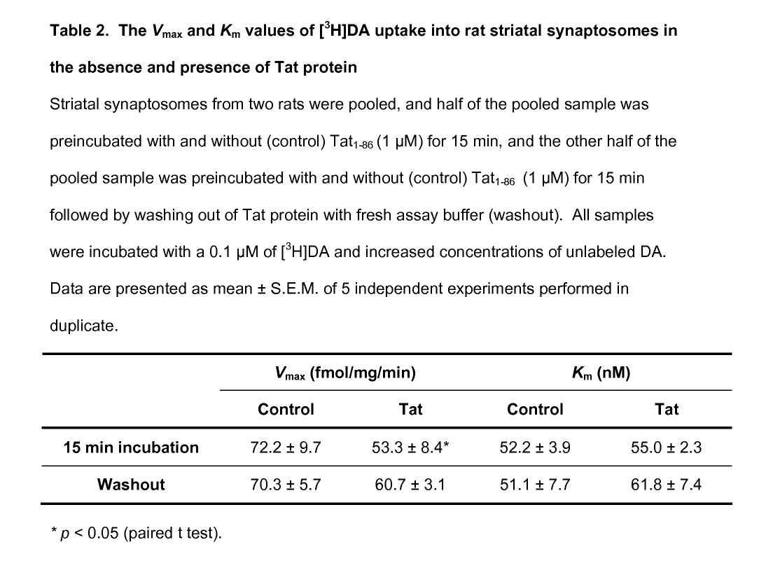

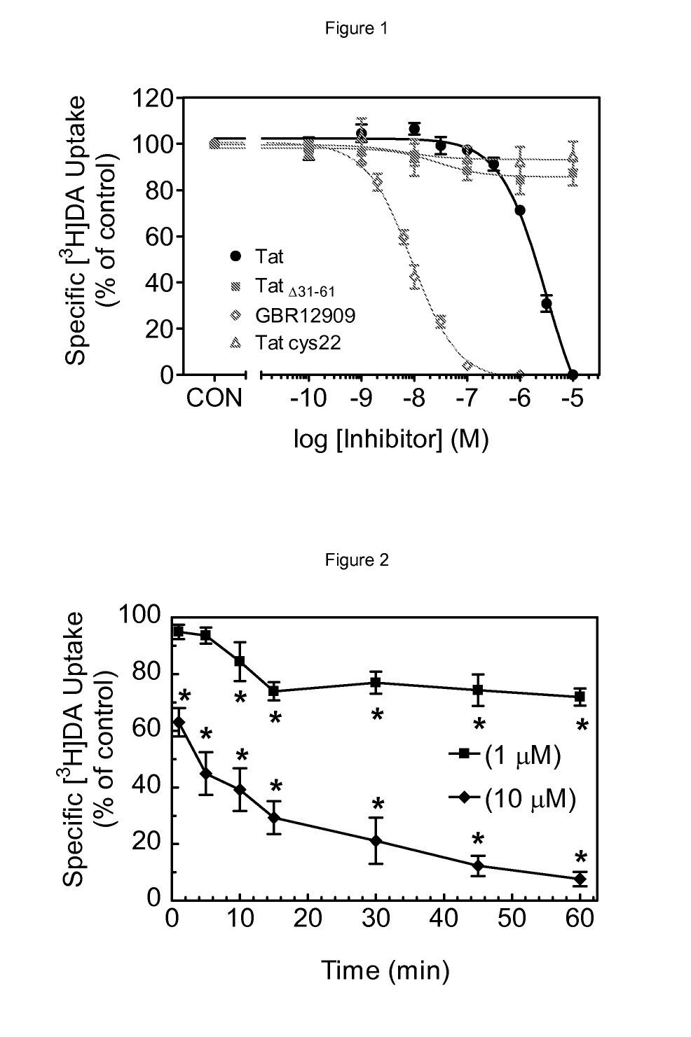

To determine the concentration-dependent inhibitory effect of Tat on specific [³H]DA uptake into the striatal synaptosomes, the competitive inhibition of [³H]DA uptake was examined in the presence of various concentrations of Tat. Specific [³H]DA uptake was substantially inhibited by Tat (Kᵢ=1160±100 nM). GBR 12909 was used as positive control for the [³H]DA uptake assay, and had a Kᵢ value of 19±1.5 nM (Table 1). Tat_{Δ31-61} and Tat cys22 were negative controls for the assay, as neither inhibited [³H]DA uptake across the concentration range of 0.1 nM to 10 μM (FIG. 1). The time course of Tat-induced inhibitory effects on [³H]DA uptake was determined at 1 to 60 min by incubation with 1 or 10 μM Tat. As seen in FIG. 2, one-way ANOVAs revealed significant time-dependent inhibition in [³H]DA uptake for Tat at 1 μM concentration (F_{7.24}=49.2, p<0.001) and 10 μM concentration (F_{7.24}=51.1, p<0.001). 10 μM Tat inhibited [³H]DA uptake by 37% at 1 min, and the inhibitory effects remained until 60 min (p<0.05, paired Student's t test).

To determine the effect of Tat on Vₘₐₓ and Kₘ values for [³H]DA uptake into striatal synaptosomes, kinetic analysis of [³H]DA uptake was performed in the absence (control) or presence of Tat (1 and 10 μM). The Eadie-Hofstee analysis of the [³H]DA uptake showed that 15 min preincubation with Tat (1 μM) significantly decreased Vₘₐₓ value by ˜27% (53.3±8.4 pmol/mg/min), compared to control (72.2±9.7 pmol/mg/min, paired t₍₃₎=13.5, p<0.01, FIG. 3A). There was no change in Kₘ value between Tat treated and control samples (55.2±2.3 and 52.2±3.9 nM, Table 2). A high concentration of Tat (10 μM) was used to determine if the inhibitory effect of Tat on the parameters (Vₘₐₓ and Kₘ) of [³H]DA uptake was concentration dependent. As shown in FIG. 3B, Tat (10 μM) caused a 10-fold decrease in Vₘₐₓ value (7.2±1.4 pmol/mg/min), compared to control (70.2±9.2 pmol/mg/min, paired t₍₃₎=7.8, p<0.001); the Kₘ was increased from 49±3.3 nM to 106±3.7 nM (paired t₍₃₎=4.64, p<0.05). Although this could therefore be regarded as an example of a mixed inhibition mechanism of the noncompetitive variant-type, the reduction in Vₘₐₓ could reflect a trafficking phenomenon rather than a particular kinetic mechanism.

The Tat-induced decrease in Vₘₐₓ was evaluated to determine whether it was reversible. For this example, paired samples of striatal synaptosomes were preincubated with Tat (1 μM) or without Tat (control) for 15 min. Subsequently, the samples were recentrifuged at 20,000 g for 15 min and the resulting pellets were resuspended with fresh assay buffer. As shown in Table 2, 15 min preincubation with Tat (1 μM) followed by Tat washout did not alter either Vₘₐₓ or Kₘ, relative to the control. These results suggest that the inhibitory effect of Tat on Vₘₐₓ is reversible.

### Tat Protein Inhibits [³H]WIN35,428 Binding.

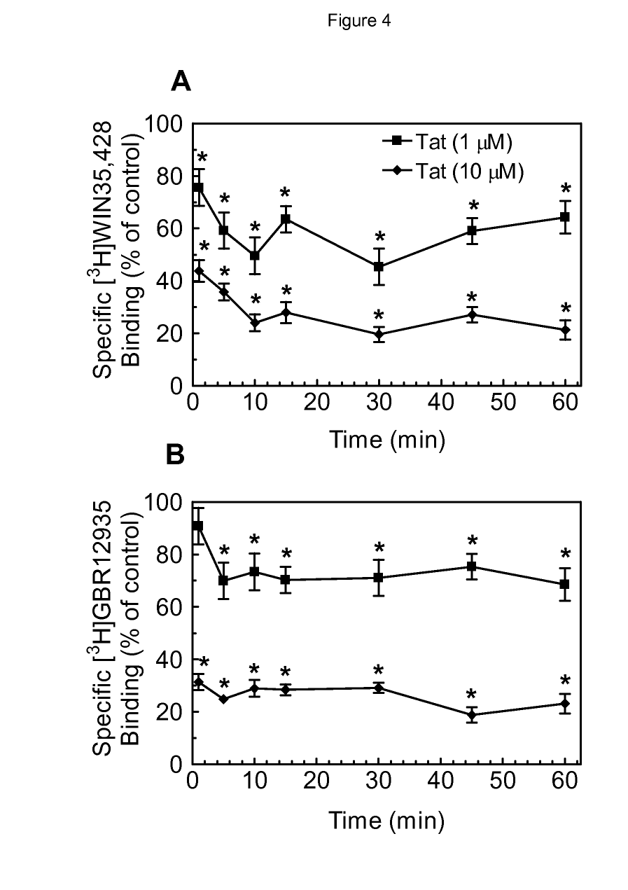

To ascertain whether the inhibitory effect of Tat on [³H]DA uptake was associated with a direct interaction of Tat protein with DAT protein, the ability of Tat protein to inhibit [³H]WIN35,428 binding was determined using striatal synaptosomes. As shown in FIG. 4A, Tat at 1 or 10 μM concentration significantly inhibited [³H]WIN35,428 binding during the 60 min period (1 μM Tat: F_{7.24}=45.6, p<0.001 or 10 μM Tat: F_{7.24}=250, p<0.001). Exposure to Tat for 1 min caused 26% and 66% reduction in [³H]WIN35,428 binding for 1 and 10 μM, respectively, and these significant inhibitory effects remained through 60 min (p<0.05, paired Student's t test). Analysis of inhibition by Tat of specific [³H]WIN 35,428 binding was undertaken to determine the interaction of Tat with the binding site for this radioligand on DAT protein. Concentration response curves for Tat protein, GBR 12909 or WIN 35,428 to inhibit [³H]WIN 35,428 binding from the striatal synaptomses are illustrated in FIG. 5A. GBR 12909 and WIN 35,429 were used to clarify different binding sites for [³H]WIN 35,428 binding on DAT protein, and they were found to have Kᵢ values of 30±2.3 and 2.81±0.14 nM, respectively (Table 1). Tat (1 μM) inhibited the specific [³H]WIN 35,428 binding with Kᵢ values of 400±39 nM (Table 1).

The effect of Tat on the Bₘₐₓ and K_{d} of [³H]WIN 35,428 binding in striatal synaptosomes was also determined in the absence or presence of Tat (0.2 and 1 μM). As shown in FIG. 6A, a low concentration of Tat (0.2 μM) significantly decreased the Bₘₐₓ value of [³H]WIN 35,428 binding by ˜18% (10.2±2.4 pmol/mg protein), compared to control (12.3±0.7 pmol/mg protein, paired t₍₃₎=4.35, p<0.05). Tat (0.2 μM) also significantly increased the K_{d} value (5.5±0.4 nM) compared to the control (2.1±0.2 nM, paired t₍₃₎=7.94, p<0.05, FIG. 6B). Similarly, Tat at 1.0 μM caused a 18% decrease in the Bₘₐₓ (12.6±2.4 nM) compared to the control (15.3±2.7 nM, paired t₍₃₎=2.83, p<0.05, FIG. 6C), with an increase in the K_{d} value (Tat: 11.2±2.8 nM, control: 2.3±0.6 nM, paired t₍₃₎=10.2, p<0.05, FIG. 6D). Although these results could be described in terms of Tat protein acting as an apparent uncompetitive inhibitor on [³H]WIN 35,428 binding, it should be kept in mind that this is only valid as a descriptive narrative regarding the appearance of the Scatchard plot (binding) in comparison with an Eadie-Hofstee plot (uptake): Application of the underlying uncompetitive mechanism to the binding situation (inhibitor binding preferentially to the transporter-ligand complex) leads to radically different kinetical outcomes compared with the substrate transport case. In addition, Bₘₐₓ changes could reflect trafficking rather than kinetic effects.

### Tat Protein Inhibits [³H[GBR 12935 Binding.

To ascertain whether the inhibitory effect of Tat on [³H]DA uptake was associated with a interaction of different binding sites with DAT protein, the ability of Tat protein inhibit [³H]GBR 12935 binding was evaluated using the striatal synaptosomes. As illustrated in FIG. 4B, results from the time course study revealed a significant time-dependent inhibition of [³H]GBR 12935 binding for Tat at 1 μM (F_{7.24}=10.3, p<0.001) and 10 μM (F_{7.24}=280, p<0.001), respectively. At 1 μM, Tat-induced inhibition on [³H]GBR 12935 binding was 25% at 5 min and then remained until 60 min. At a high concentration (10 μM), Tat inhibited [³H]GBR 12935 binding by ˜77% throughout the 60 min period (p<0.05, paired Student's t test).

Analysis of inhibition by Tat of specific [³H]GBR 12935 binding was undertaken to determine the interaction of Tat with the binding site for this radioligand on DAT protein. Concentration response curves for Tat protein, GBR 12909 or WIN 35,428 to inhibit [³H]GBR 12935 binding from striatal synaptosomes are illustrated in FIG. 5B. GBR 12909 and WIN 35,428 were used to distinguish different binding sites for [³H]GBR 12935 binding on DAT protein, and they showed Kᵢ values of 1.2±0.2 and 17.9±4.4 nM, respectively (Table 1). Tat (1 μM) inhibited the specific [³H]GBR 12935 binding with Kᵢ values of 1610±150 nM (Table 1).

The effect of Tat on the Bₘₐₓ and K_{d} of [³H]GBR 12935 binding in striatal synaptosomes was determined in the absence or presence of Tat (0.2, 1.0 or 10 μM). As shown in FIG. 7A, a low concentration of Tat (0.2 μM) did not inhibit either the Bₘₐₓ or K_{d} values of [³H]GBR 12925 binding. Tat (1 μM) decreased the Bₘₐₓ value by ˜17% (57.9±7.3 pmol/mg protein), compared to the control (70.1±9.7 pmol/mg protein, paired t₍₄₎=2.94, p<0.05), with no change in the K_{d} value (FIG. 7B). At a high concentration (10 μM), Tat caused a 60% reduction in the Bₘₐₓ (22.4±4.3 pmol/mg protein) compared to the control (55.4±5.7 pmol/mg protein, paired t₍₄₎=5.6, p<0.05), with no change in the K_{d} value (FIG. 7C). These results could be interpreted to suggest that Tat protein inhibits [³H]GBR 12935 binding in a noncompetitive manner, but, again, this is only true descriptively: Actual noncompetitiveness in the binding situation (as opposed to the transport case) would not result in any change in binding as inhibitor binds equally well to the free or ligand-complexed transporter. Additionally, Bₘₐₓ changes could reflect trafficking rather than kinetic effects.

Effect of Tat Protein on [³H]Leucine Uptake into Rat Striatal Synaptosomes.

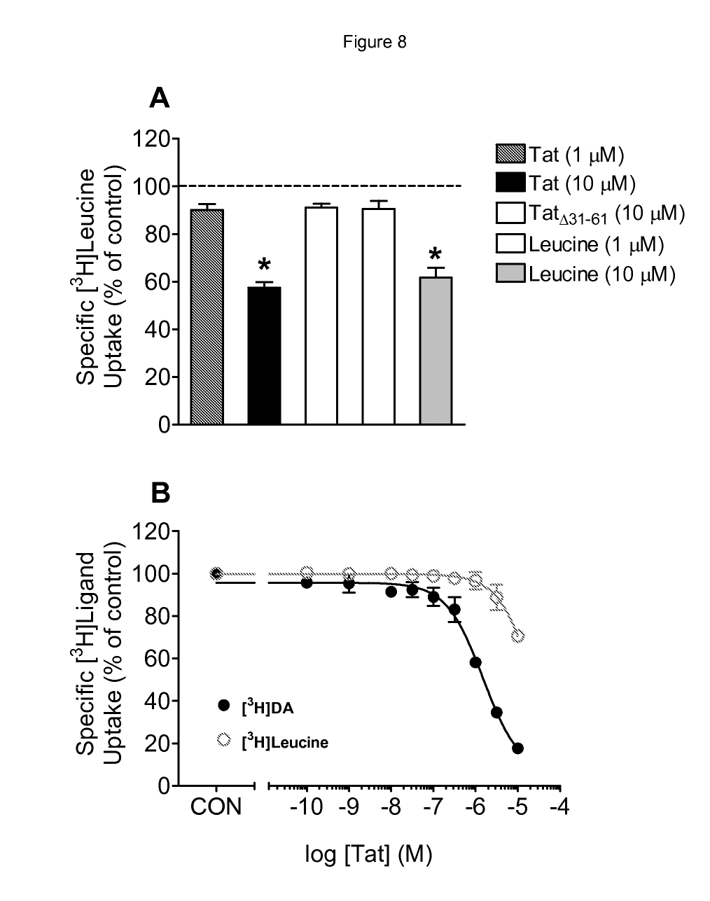

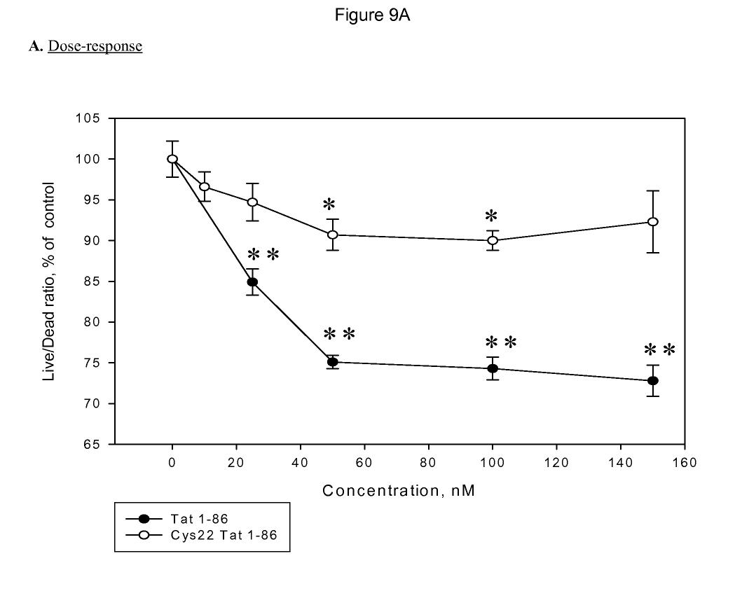

The potential inhibitory effect of Tat on electrochemical gradients was determined by monitoring [³H]leucine uptake, which, as DA uptake, depends on membrane potential and ion gradients. Tat_{Δ31-61} and leucine were used as the negative and positive control, respectively. As illustrated in FIG. 8A, 10 μM Tat_{Δ31-61}, Tat (1 μM) or leucine (1 μM) showed a similar inhibition (˜10%) on [³H]leucine uptake. At a concentration of 10 μM, either Tat or leucine caused about 40% decrease in [³H]leucine uptake (p<0.05, paired Student's t test). To reduce the Tat effect on the electrochemical gradients, a separate experiment was performed with reduced exposure time to Tat (FIG. 8B). Under these conditions, Tat inhibited [³H]DA uptake with a Kᵢ value of 0.51±0.01 μM, which was 100-fold higher than that for [³H]leucine uptake (K=56±6.7 μM, FIG. 8B). 1 μM Tat had no effect on the [³H]leucine uptake, whereas 10 μM Tat inhibited the [³H]leucine uptake by 30%. A clear separation of inhibition curves for Tat was observed between the [³H]DA and [³H]leucine uptake assays (FIG. 8B). These results indicate that at a low concentration of 1 μM, the inhibition of [³H]DA uptake by Tat as shown in FIG. 1 was not attributable to alterations in electrochemical gradients.

Further, the effect of the mutation of the cysteine 22 was investigated, which is essential for Zn⁺²-chelating and trans-activation abilities of HIV-1 Tat, with Gly on neurotoxic properties of the recombinant Tat 1-86 (clade B) in the primary culture of rat fetal hippocampal neurons.

Recombinant original Tat 1-86 and (Cys22→Gly22)-substituted Tat 1-86 (LAl/Bru strain of HIV-1 clade B, GenBank accession no. K02013) were purchased from Diatheva (Italy). Tat 1-101 clade C was purchased from Prospec (Israel).

Primary hippocampal cell cultures were prepared from 18-day-old Sprague-Dawley rat fetuses as previously described in Aksenov M Y, Aksenova M V, Nath A, Ray P D, Mactutus C F, Booze R M, 2006, Cocaine-mediated enhancement of Tat toxicity in rat hippocampal cell cultures: the role of oxidative stress and D1 dopamine receptor, Neurotoxicology, 27, 217-228, which is incorporated by reference herein. Cultures were used for experiments after 12 days in culture and were >85-90% neuronal as determined by anti-MAP-2/anti-GFAP/Hoechst fluorescent staining.

The treatment of hippocampal cell cultures was carried out by the addition of freshly-prepared stock solutions of the recombinant Tat polypetides into the cell culture growth medium. Equal volume of the vehicle was added to control cell cultures. To determine dose-response neurotoxicity curves, groups of individually grown cell cultures were exposed to 10-150 nM concentrations of Tat or Cys22 Tat for 48 hours. For the time course experiments, cell cultures were continuously incubated with 50 nM Tat or 50 nM Cys22 Tat for 2, 24, 48, and 96 hours. To study binding/uptake, 50 nM Tat or 50 nM Cys22 Tat was added to the cultured cells and the incubation was carried out for different time periods from 1 min to 2 hours. Cytotoxic effects induced by different time periods (1 min-2 hours) of transient exposure of hippocampal cultures to Tat- or Cys22 Tat were studied as described in Aksenova M V, Aksenov M Y, Mactutus C F, Booze R M, 2009, Neuronal survival and resistance to HIV-1 Tat toxicity in the primary culture of rat fetal neurons, Exp Neurology, 215, 253-263, which is incorporated by reference herein. For the comparison of the neurotoxic effects of Cys 22 Tat 1-86, Tat 1-86 (clade B), and Tat 1-101 (clade C), hippocampal cultures were exposed for 48 hours to 100 nM dose of the either one of recombinant Tat polypeptides.

Neuronal survival was determined using a Live/Dead viability/cytotoxicity kit from Molecular Probes (Eugene, Oreg.) in rat fetal hippocampal cell cultures prepared in 96-well plates as described in above. Fluorescence was measured using a Bio-Tek Synergy HT microplate reader (Bio-Tek Instruments, Inc., Winooski, Vt.).

Binding/uptake of Tat and Cys22 Tat was studied using anti-Tat immunocytochemistry in acetic alcohol-fixed cultures, immunobloting of cell lysates, and the direct ELISA measurements of concentrations of Tat polypeptides in the cell culture growth medium. Rabbit polyclonal anti-Tat antibody (Diatheva, Italy) that recognizes both Tat and Cys 22 Tat immunoreactivities was used in these experiments.

Fluorochrome Inhibitor of Caspases (FLICA) Caspase 9 (red fluorescence) or Caspase 3/7 (green fluorescence) Apoptosis Detection Kit (Immunochemistry Technologies LLC, Bloomington, Minn.) were used to detect active caspases in cultures exposed to Tat and Cys22 Tat. Hoechst staining (blue fluorescence) was used to label cell nuclei. Microplate reader-based analysis and fluorescence microscopy imaging of specific fluorescence of SR-LEND or FAM-DEVD complexes with active caspase 9 or 3/7 were carried out as described in above.

Statistical comparisons were made using ANOVA and planned comparisons were used to determine specific treatment effects. Significant differences were set at P<0.05.

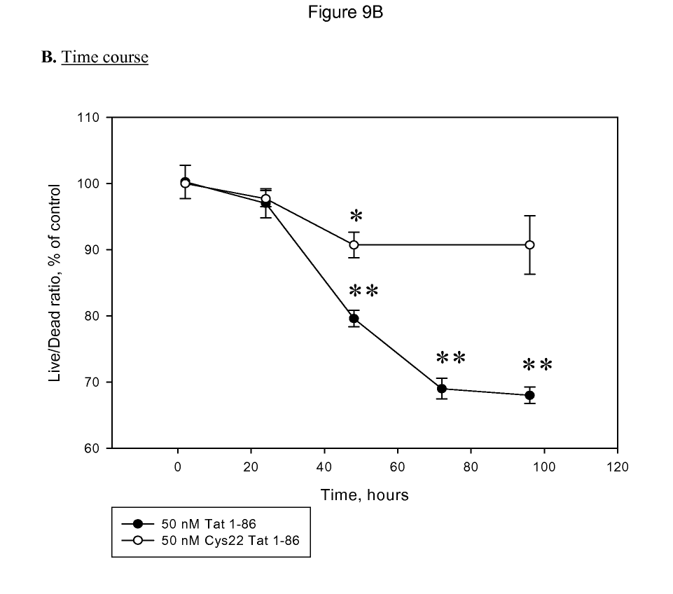

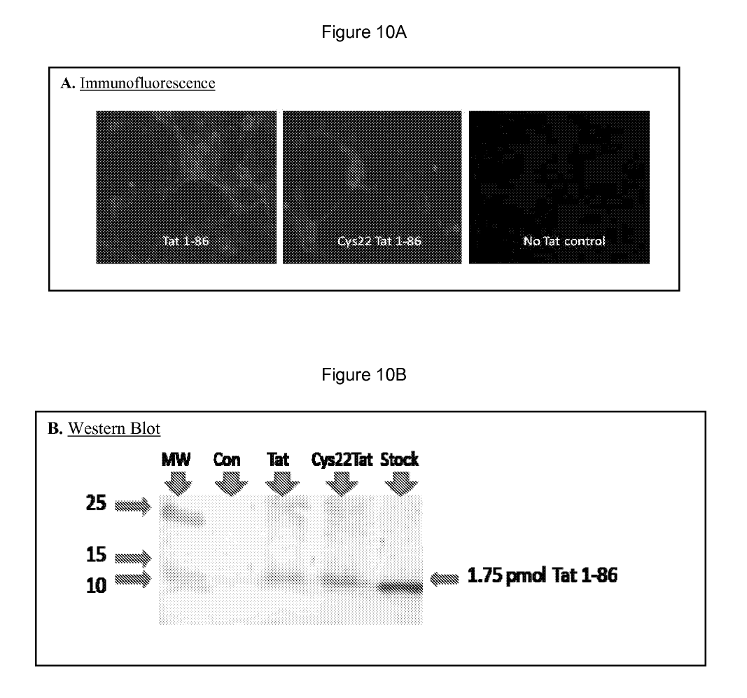

The dose-response curve of Tat 1-86 toxicity is shown in FIG. 9A. The analysis of dose-response curves demonstrated that Tat 1-86 was significantly (P<0.05) more toxic to hippocampal cell cultures than Cys22 Tat 1-86 following 48 hours of treatment.

It could be argued that the substitution of cysteine 22 in Tat 1-86 simply delayed the development of Tat-mediated neurotoxicity. However, the comparison of the toxicity time courses in Tat—and Cys22 Tat—(FIG. 9B) did not favor this suggestion since even the prolonged exposure to cysteine 22-substituted Tat variant failed to induce more than 10% decrease in cell viability. At 96-hour time point the Live/Dead ratio in Cys22 Tat-treated cultures was 90.7±4.43% of control and this difference in cell viability did not reach statistical significance (P>0.05) due to high variation of individual numbers.

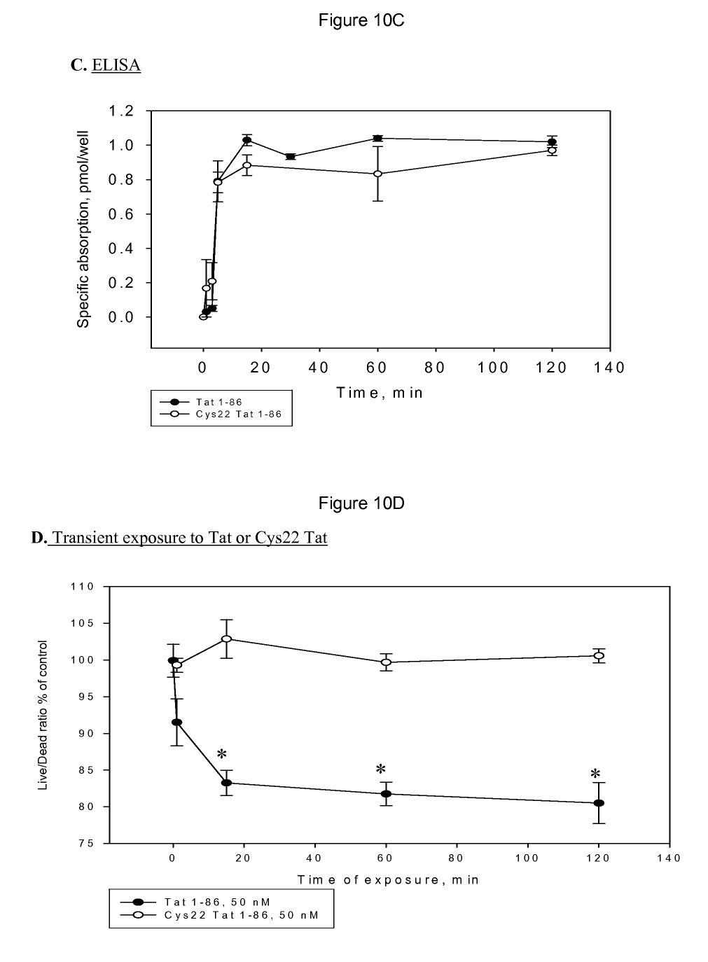

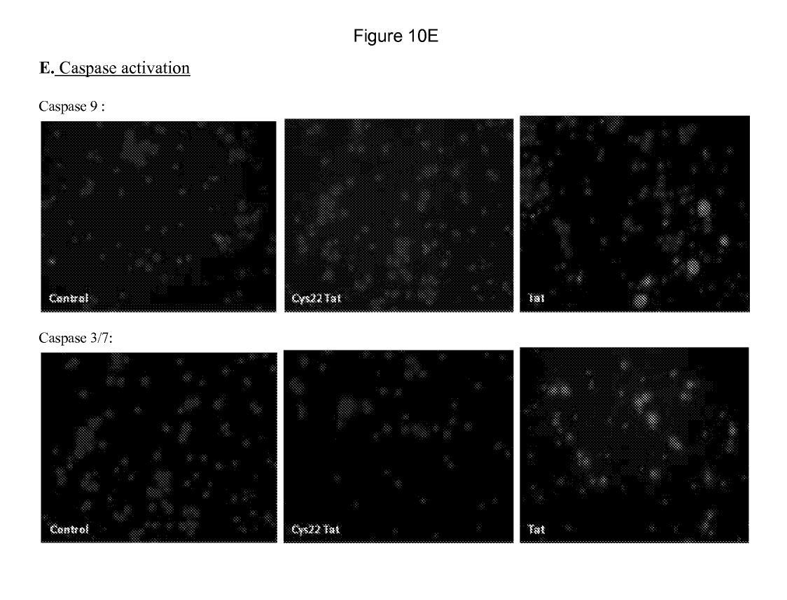

Binding to the cell membrane is the key step in the process of Tat neurotoxicity. The cysteine 22 mutation does not affect intracellular distribution of the extracellular Tat protein following its internalization. Nevertheless, it was still possible that altered binding/uptake properties could be a reason for the attenuated neurotoxic ability of Cys22-substituted Tat 1-86. The presence of cell-bound/internalized Tat 1-86 or Cys22 Tat 1-86 Tat in hippocampal neurons after 2-hour exposure to 50 nM dose to either one of Tat variants was evident by immunofluorescent microscopy and Western blotting (FIG. 10A, B). The curves describing the specific absorption of Tat immunoreactivity by hippocampal cells in Tat 1-86- and Cys22 Tat 1-86-exposed cultures were not significantly different from each other (FIG. 10C). Neurotoxic effects produced by the transient exposure of cell cultures to 50 nM Tat 1-86 were time-dependent and consistent with the kinetic of Tat absorption in hippocampal cultures (FIG. 10D). In contrast, transient exposure of cultures to 50 nM Cys22 Tat 1-86 was unable to cause cytotoxicity. Thus, it can be concluded that the impaired toxicity of Cys22 Tat 1-86 was not attributable to any noticeable changes in its ability to interact directly with neurons.

The variant of Tat protein from HIV-1 clade C (Tat 1-101, clade C) was believed to be less neurotoxic than its analogs from HIV-1 clade B strains. More than 90% of HIV-1 clade C viruses encode Tat with serine at the position 31 instead of cysteine 31. Therefore, it was determined that the toxicity of the equal dose of Tat clade B, Cys22-substituted Tat clade B and Tat clade C in hippocampal cell cultures. The 100 nM final concentration is the dose the recombinant Tat clade B, which produces maximum cell viability decrease (Live/Dead ratio: 75±2.6% of control) in hippocampal cell cultures. The Live/Dead ratios in groups of cultures treated with Cys22 Tat clade B or Tat clade C were 90±1.4% vs control and 95±3.0%, subsequently. Comparisons of cell cultures groups exposed to either Cys22 Tat 1-86 or Tat 1-101 (clade C) demonstrated that both Tat variants were significantly (P<0.05) less neurotoxic than Tat 1-86 (clade B). The cell viability changes induced by 48-hour treatment of cell cultures with equal doses of either Cys22 Tat 1-86 or Tat 1-101 (clade C) were not significantly different (P>0.05). If indeed the attenuated neurotoxicity of HIV-1 C Tat is attributed to the conservative Cys31 mutation, the results of the present disclosure imply that point-mutation of two different cysteine residues within the Cys-rich domain may similarly impair the ability of Tat to cause neuronal degeneration.

Extracellular Tat induces caspase activation in primary cultures of rat fetal neurons. Consistently, the increased activity of the initiator caspase 9 has been detected following the 2-hour exposure of hippocampal cultures to 50 nM Tat 1-86 (129.7±4.4% SR-LEND red fluorescence vs vehicle-treated control, n=7). Activation of the effector caspase 3/7 (179.8±4.5% FAM-DEVD green fluorescence vs vehicle-treated control, n=8) was determined in hippocampal cultures after the 24-hour exposure to 50 nM Tat 1-86. Consistent with the results of the microplate reader-based measurements of caspase 9 and 3/7 activities, increased numbers of SR-LEND and FAM-DEVD-positive cells in Tat-treated cultures were observed using the fluorescent microscopy. Under the standard image acquisition conditions, 9-15 SR-LEND- positive cells per hundred of Hoechst-labeled cells were typically found in hippocampal cultures, which were exposed to 50 nM Tat 1-86 for 2 hours and 17-20 cells out of a hundred of Hoechst-stained cells were FAM-DEVD-positive following 24-hour Tat exposure. On the contrary, neither method of the analysis of SR-LEND and FAM-DEVD fluorescent signals have detected increased caspase activities in cultures exposed to 50 nM Cys22 Tat 1—(FIG. 10E).

Results of the present disclosure clearly demonstrate that the mutation of cysteine 22, which is a part of the putative HIV-1 Tat metal-binding site, reduces the ability of the recombinant Tat 1-86 (clade B) to initiate apoptotic cascades in hippocampal neurons. The present disclosure is in agreement with the suggestion that zinc binding plays important role in apoptosis induced by extracellular Tat in mammalian cells.

Interactions with various membrane-integrated proteins play a key role in the mechanism of neuronal degeneration induced by extracellular Tat. At the cell membrane level, cysteines involved in metal-chelating (particularly Zn⁺²-chelating) may be essential for Tat ability to alter functioning of neuronal receptor and transporter proteins and trigger the downstream cell death signaling. There is evidence indicating that the Cys22 mutation in Tat 1-86 makes it unable to interact with dopamine transporter and inhibit dopamine uptake in dopaminergic cells. It is possible that the substitution of cysteine 22 may impair the ability of Tat to interfere with the Zn²⁺-mediated control of NMDAR activity and thereby attenuate Tat pro-apoptotic effects in hippocampal neurons. An important role of Tat actions at the NMDA receptor's Zn⁺² binding site for the ability of Tat to potentiate NMDA-induced currents has been suggested. Extensive stimulation of NM DAR can trigger the intrinsic apoptotic pathway mediated by the release of mitochondrial cytochrome c and activation of caspases 9 and 3. Targeting of cysteines that form Tat metal-binding site may be considered as one of the therapeutic strategies to simultaneously block transactivating and neurotoxic Tat abilities in NeuroAIDS.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed:

1. A method for increasing dopaminergic neurotransmission in a mammal in need of such treatment comprising disrupting Tat-DAT binding in the mammal.

2. The method of claim 1, wherein the method treats HIV triggered cognitive-motor disorders.

3. The method of claim 1, wherein the method alters one or more of proteins comprising Tat_{Δ31-61} and Tat cys22.

4. The method of claim 1, wherein the method alters protein Tat_{Δ31-61}.

5. The method of claim 1, wherein the method alters protein Tat cys22.

6. The method of claim 1, wherein the method further comprises treatment for one or more addictive substances.

7. The method of claim 6, wherein the one or more addictive substances comprise cocaine.

8. The method of claim 6, wherein the one or more addictive substances comprise amphetamine.

9. The method of claim 1, wherein the method reduces the neurotoxicity of Tat.

10. The method of claim 1, wherein the method is administered in vivo.

11. The method of claim 1, wherein the method further comprises monitoring DAT function.

12. The method of claim 1, wherein the mammal is a human.

13. A method for increasing dopaminergic neurotransmission in a mammal in need of such treatment comprising disrupting Tat-DAT binding in the mammal by altering protein Tat cys22.

14. The method of claim 13, wherein the method treats HIV triggered cognitive-motor disorders.

15. The method of claim 13, wherein the mammal is infected with HIV-1 clade C.

16. The method of claim 13, wherein the method further comprises altering protein Tat_{Δ31-61}.

17. The method of claim 13, wherein the method further comprises treatment for one or more addictive substances.

18. The method of claim 17, wherein the one or more addictive substances comprise cocaine.

19. The method of claim 18, wherein the one or more addictive substances comprise amphetamine.

20. The method of claim 13, wherein the method reduces the neurotoxicity of Tat.

21. The method of claim 13, wherein the method is administered in vivo.

22. The method of claim 13, wherein the mammal is a human.
